(19) United States
(12) Patent Application Publication
(10) Pub. No.: US 2011/0263919 A1
(43) Pub. Date: Oct. 27, 2011

(54) Chemical Reactor Operation

(75) Inventor: David James West, Ducklington (GB)

(73) Assignee: CompactGTL plc, Abingdon Oxfordshire (GB)

(21) Appl. No.: 13/133,020

(22) PCT Filed: Dec. 2, 2009
(86) PCT No.: PCT/GB2009/051634

§ 371 Date: Jun. 30, 2011

(30) Foreign Application Priority Data
Dec. 11, 2008 (GB) 0822544.3

(51) Int. Cl. C07C 5/00; B01J 8/00

(52) U.S. Cl. 585/700; 422/600

(57) ABSTRACT

A method of operation of one or more chemical reactors, wherein each reactor defines first flow channels for a chemical reaction process in proximity to second flow channels for heat transfer, and each reactor is provided with fluid connections for bringing about flows of respective fluids through the first and second flow channels, involves the steps of shutting down the flows of fluids through at least one of the first and second flow channels, and then changing the fluid connections, and then reopening the fluid connections. There is no change in the chemical reaction process performed by the reactors. The change to the fluid connections is preferably such as to achieve a flow reversal. This may involve turning the reactor itself around, or changing the arrangement of ducts connected to the reactor. This changes the thermal stress distribution within the reactor, and can consequently increase the reactor's operational lifetime.

### CROSS-REFERENCE TO RELATED APPLICATION

This application is a continuation of PCT Application No. PCT/GB2009/051634, filed Dec. 2, 2009 and claiming priority to GB Application Nos. 0822544.3 filed Dec. 11, 2008, the disclosure of which is incorporated herein by reference in its entirety.

### DESCRIPTION

This invention relates to a method of operation of one or more chemical reactors so as to increase the operational lifetime of the or each reactor, and to a reactor provided with means to increase the operational lifetime of the reactor by such a method.

The operational lifetime of a reactor is influenced by the stresses under which it is operated. The stress that a reactor can tolerate is dependent on the temperature at which the reactor is operated. Depending on the reaction or reactions taking place within a reactor thermal stresses may not be uniform throughout a reactor. The reactor must be replaced as soon as any part of the reactor requires replacement, even though a proportion of the reactor may still in a condition in which it could continue to be operated for some considerable time.

Some chemical reactors are operated in remote locations. For example, reactors used for processing associated gas may be operated in the vicinity of oil wells from which the associated gas is drawn. The reactors used in such locations may include, but are not limited to, syngas generating reactors, generating synthesis gas by steam methane reforming, autothermal reforming or partial oxidation, or by using ion transfer membranes; and Fischer-Tropsch synthesis reactors that produce syncrude from the syngas. When processing associated gas, it is important to process the gas as it is produced and to minimize reactor downtime when gas cannot be processed. If the gas cannot be processed, then it may have to be flared, and the penalties for flaring are increasingly severe.

It has previously been suggested that the catalysts required for the reactions outlined above may be provided coated onto the walls of the reactor. In this case, the catalyst life may limit the life of the reactor. More recently it has been suggested that the catalyst may be provided within reaction channels on removable inserts such as, for example, foils. As a result the catalyst life no longer limits the reactor life. Instead, during the reactor's life there are periodic shutdowns to replace the catalyst.

Many chemical reaction processes, including the syngas generation and Fischer [symbol] Tropsch synthesis reactions mentioned above, necessitate heat transfer either to or from the chemical reactants. Chemical reactor designs have been proposed that define many first channels, for a chemical reaction process, and many second channels, for providing or removing heat. Since the channels can be close together, and separated only by an intervening wall, such a design can provide good heat transfer between the first and second channels. By way of example such a reactor may be formed from a stack of plates that are arranged to define the first and the second flow channels alternately in the stack, the stack being bonded together. In those channels in which a chemical reaction is to occur a catalyst may be provided either on the walls of the flow channel or on a catalyst-carrying insert inserted into the channel. However there are temperature differences within such a reactor between the first channels and the second channels, and indeed there are almost always significant temperature differences along the length of any one channel, so that the mechanical stresses arising from thermal expansion are non-uniform. These thermally-generated stresses can, depending on the temperature of the reactor material, reduce the operational lifetime of the reactor.

In some reactor systems, in order to provide each reactor with the fluids that flow in each of the first and second channels, headers are provided. The input and output flows from the reactors are linked by ducting that links the output from a first stage reactor with the input of a second stage reactor etc. In order to control the flows, valves may also be provided. The configuration of the headers, valves and ducting results in each reactor having a unique position within a system. In some reactor systems, the reactors are held within a pressure vessel and in this case the pressure vessel itself may take the place of one of the headers. The present invention is equally applicable to both these and other types of reactor.

The present invention has been devised in order to address and mitigate some or all of the above mentioned problems.

According to the present invention there is provided a method of operation of one or more chemical reactors, wherein each chemical reactor defines first flow channels for a chemical reaction process in proximity to second flow channels for heat transfer, and each chemical reactor is provided with fluid connections for bringing about flows of respective fluids through the first flow channels and the second flow channels, wherein the method comprises modifying the flows of fluid through the first flow channels or the second flow channels or both, so as to change the temperature distribution within the or each reactor, while the chemical reaction process that takes place in the chemical reactors remains substantially the same.

Preferably the method comprises the steps of shutting down the flows of fluids through at least one of the first flow channels and the second flow channels, and then changing the fluid connections, and then reopening the fluid connections.

By changing the fluid connections between successive uses of the reactor, the temperature distribution within each channel is altered and hence the thermal stress and material temperature distribution within the reactor is altered. The region of the reactor in which the thermal stresses are greatest is thereby changed, and as a consequence the operational lifetime of the reactor may be increased. There are several different ways in which the fluid connections may be changed.

The modification to the fluid connections is preferably applied during maintenance or shutdown of a plant that includes the chemical reactor, or during maintenance or shutdown of the chemical reactor. In a first example a reactor is disconnected from inlet and outlet pipes or ducts, and the reactor is then turned around, and the pipes are then reconnected so that the flow direction through the reactor is reversed. In an alternative, after a reactor is disconnected, pipes or ducts constituting the fluid connections are altered and then reconnected so that the flow direction through the reactor is reversed in either the first flow channels or the second flow channels, or both. Where two reactors are used in series for performing a two-stage reaction, then the first and second stage reactors may be exchanged. In some situations it may be possible to exchange the fluid flows to the first and second channels, so that during the next stage of operation the chemical reaction occurs in the second flow channels.

It must be appreciated that the second flow channels—that is to say the flow channels for heat transfer—may contain a heat exchanging fluid; or alternatively they may contain a fluid mixture that undergoes a second chemical reaction. For example if the chemical reaction process in the first flow channels is endothermic, the requisite heat may be supplied either by a hot fluid such as exhaust gases, in the second flow channels, or alternatively by performing an exothermic reaction such as combustion in the second flow channels. On the other hand if the chemical reaction process in the first flow channels is exothermic, the requisite removal of heat from the first flow channels may be achieved by supplying a coolant fluid through the second flow channels, or by performing an endothermic chemical reaction in the second flow channels.

Where a plant comprises a plurality of chemical reactors in all of which the same chemical reaction is performed, the reactors may all be in parallel, or all in series, or may be arranged as parallel sets of series reactors. Where chemical reactors operate in parallel, then the plant can continue to operate while one or more of the chemical reactors are shut down. The present invention is especially applicable to the one or more of the chemical reactors that are shut down, while the remainder of the plant continues to operate. However, it will also be understood that the present invention is also applicable to single reactor systems.

The present invention also provides a chemical plant comprising one or more chemical reactors and incorporating means to enable the said method of operation to be carried out.

In a case where the reactor comprises first and second flow channels in which endothermic and exothermic reactions occur, respectively, and the modification to the flows of fluids involves exchanging the flows to the first and second flow channels, then the reactor may have first flow channels and second flow channels that have substantially the same dimensions. This ensures that the catalyst inserts that are provided for the channels in the first configuration can be equally well accommodated in the second configuration, so that the method can also involve exchanging the catalyst inserts between the first and second flow channels. (However, if the catalysts in the channels are the same, there is no need to exchange them.) Having channels of the same dimensions also ensures that the same reaction volumes and heat transfer conditions exist in each configuration so that the reactor will behave in substantially the same way regardless of which configuration is being employed. Thus, in this context, a different chemical reaction is carried out in each set of flow channels before and after the flow modification, although the reactor as a whole continues to perform the same chemical reaction process.

Furthermore, according to the present invention there is provided a module comprising a first reactor and a second reactor, each reactor having first flow channels and second flow channels, and ducts configured to take outputs from the first reactor to provide inputs to the second reactor, and bypass ducts and valves configured to take outputs from the second reactor to provide inputs to the first reactor. The provision of a module comprising two reactors and the ducting and valves to enable the reversal of flow minimises the work that needs to be carried out on site in order to achieve the reversal of the flows through the first reactor and the second reactor. Thus, in this case, the effect of the operation is to change which reactor carries out which stage of a two [symbol] stage process; but the chemical reaction process carried out by the module is unchanged.

The present invention is applicable to any reactor in which there are a multiplicity of reaction channels. The reactor itself may comprise a stack of plates. For example, first and second flow channels may be defined by grooves in respective plates, the plates being stacked and then bonded together. Alternatively the flow channels may be defined by thin metal sheets that are corrugated or castellated and stacked alternately with flat sheets; the edges of the flow channels may be defined by sealing strips. As another alternative flow channels may be defined by flat sheets spaced apart by spacer bars. To ensure the required good thermal contact both the first and the second flow channels may be between 10 mm and 0.5 mm high (in the heat flow direction); and each channel may be of width between about 1 mm and 50 mm. The stack of plates forming the reactor block would be bonded together for example by diffusion bonding, brazing, or hot isostatic pressing. The nature of the first and second flow channels would depend upon the reaction or reactions that are to occur in the reactor. For example channels for an exothermic chemical reaction may be arranged alternately in the stack with channels for an endothermic reaction; in this case appropriate catalysts would have to be provided in each channel. For example the exothermic reaction may be a combustion reaction, and the endothermic reaction may be steam methane reforming. In other cases channels for a chemical reaction (first channels) may be arranged alternately in the stack with channels for a heat transfer medium, such as a coolant. In this case a catalyst would only be required in the first channels. For example the first channels may be for performing the Fischer-Tropsch reaction, and the heat transfer medium would in this case be a coolant. If the catalyst is to be provided on a removable insert, the channels that contain the catalyst are preferably at least 2 mm high and at least 2 mm wide. The invention is applicable to other reactor types, and as an alternative the reactor may comprise a shell and tubes.

Where a removable insert is provided as catalyst carrier, this may comprise one or more corrugated foils. The catalyst might instead be provided on meshes, foams, or felts. In each case the catalyst carrier may form part of the reactor structure, or may be non-structural. Alternatively the catalyst may be provided on the internal surfaces of the channels. In some cases the catalyst may be in the form of pellets.

The invention will now be further and more particularly described, by way of example only, and with reference to the accompanying drawings in which:

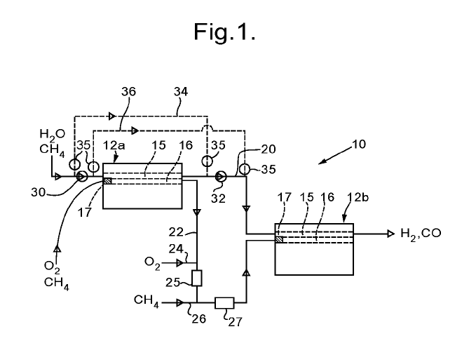

FIG. 1 shows a diagrammatic view of a two-stage steam methane reforming reactor module; and

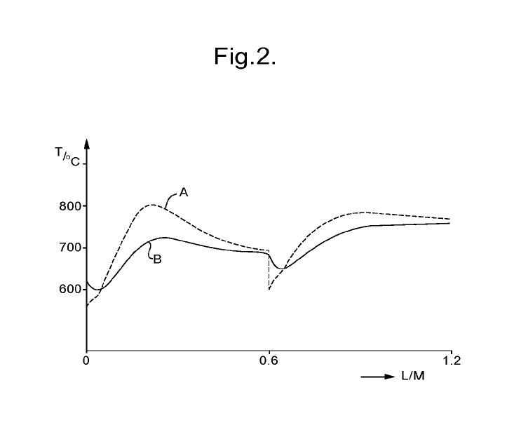

FIG. 2 shows graphically the temperature variations within the reactor module of FIG. 1.

Referring now to FIG. 1 there is shown a reaction module 10 suitable for use as a steam reforming reactor. The reaction module 10 consists of two reactor blocks 12a and 12b each of which consists of a stack of plates that are rectangular in plan view, each plate being of corrosion resistant high-temperature alloy. Flat plates are arranged alternately with castellated plates so as to define flow channels between opposite ends of the block 12a or 12b, each channel having a length 600 mm over which reaction can occur. All the channels extend parallel to each other, there being headers so that a steam/methane mixture can be provided to a first set of channels 15 and an air/methane mixture provided to a second set of channels 16, the first and the second channels alternating in the stack (the channels 15 and 16 being represented diagrammatically), such that the top and bottom channels in the stack are both combustion channels 16. Appropriate catalysts for the respective reactions may be provided on corrugated foils (not shown) in the channels 15 and 16. A flame arrestor 17 is provided at the inlet of each of the combustion channels 16. The reactor blocks 12a and 12b are shown somewhat diagrammatically, and in particular the header arrangements at each end are not shown.

A steam/methane mixture is arranged to flow through the reactor blocks 12a and 12b in series, there being a duct 20 connecting the outlet from the channels 15 of the first reactor block 12a to the inlet of the channels 15 of the second reactor block 12b. Similarly the combustion mixture also flows through the reactor blocks 12a and 12b in series, there being a duct 22 connecting the outlet from the channels 16 of the first reactor block 12a to the inlet of the channels 16 of the second reactor block 12b. The duct 22 includes an inlet 24 for additional air, followed by a static mixer 25, and then an inlet 26 for additional fuel, followed by another static mixer 27.

In use of the reaction module 10, the steam/methane mixture is preheated and supplied to the reaction module 10. A mixture of 80% of the required air and 60% of the required methane (as fuel) is preheated and is supplied to the first reactor block 12a. The temperature rises as a result of combustion at the catalyst. The outflowing hot gases are mixed with the remaining 20% of the required air (by the inlet 24 and the static mixer 25), and then with the remaining 40% of the required methane (by the inlet 26 and the static mixer 27), and supplied to the combustion channels 16 of the second reactor block 12b.

Referring now to FIG. 2, this shows graphically the variations in temperature T along the length L of the combustion channels 16 (marked A), and that along the reforming channels 15 (marked B). The portion of the graph between L=0 and L=0.6 m corresponds to the first reactor block 12a, while the portion of the graph between L=0.6 m and L=1.2 m corresponds to the second reactor block 12b. It will be noted that the temperature T in a reforming channel 15, once combustion has commenced, is always lower than the temperature T in the adjacent combustion channel 16. The combustion gas temperature undergoes a downward step change as a result of the added air (from inlet 24) between the first reactor block 12a and the second reactor block 12b (at position L=0.6 m).

It will be understood that by adjusting the space velocities in the combustion channels and in the reforming channels, and adjusting the proportion of fuel and of air provided for combustion to each reactor block, the temperature distribution through the reactor blocks 12a and 12b can be modified. The temperature variations, in particular the temperature differences between the first and second flow channels, and the temperature variations along the length of the channels, are such that thermal stresses occur in the structure of the reactor block; although the thermal stresses can be reduced by modifying the temperature distribution, they cannot be eliminated. It will also be appreciated that the temperature variations, in this example, are greater in the first stage reactor block 12a than in the second stage reactor block 12b.

The reaction module 10 may form part of a chemical plant, the synthesis gas produced by the reaction module 10 then being fed to other reactors in the plant to produce other products. The plant may incorporate a plurality of such reaction modules 10 arranged in parallel, so that the production of synthesis gas can be adjusted by changing the number of reaction modules 10 that are in use. In this case a module may be closed down, for example for maintenance, without closing down the remainder of the plant. In any event, whether there is a single such reaction module 10 or a plurality of reaction modules 10, it is occasionally necessary to close down a reaction module 10 for maintenance or servicing, for example to replace spent catalysts. When a reaction module 10 is closed down, this provides an opportunity for making changes in accordance with the present invention.

For example one reactor block, say the reactor block 12a, may be disconnected from its associated inlet and outlet ducts, and the reactor block 12a may then be turned around, and the ducts then reconnected so that the flow direction through the reactor block 12a is reversed. Alternatively it may be more convenient to leave the reactor block, say the reactor block 12a, in position, and after a reactor block 12a has been disconnected, the associated inlet and outlet ducts may be extended and connected so that the flow direction through the reactor block 12a is reversed. In this example it is preferable to make these changes to the ducts communicating with both the first flow channels 15 and the second flow channels 16, so as to ensure that the flows continue to be co-current. In other reactors it may be preferable to make such changes to only one of the sets of channels. It will be appreciated that such a change may be made to the other reactor block 12b, either instead of or as well as making the change to the reactor block 12a. Where the flow direction through a combustion channel 16 is reversed, the flame arrester 17 may be moved from one end of the channel 16 to the other; or alternatively flame arresters 17 may be provided at both ends of the channel 16.

The invention is applicable not only to a reactor in which heat is provided by catalytic combustion within the heat transfer channels, but is also applicable to a reactor in which heat is provided by hot gases produced by an external combustion reaction, the hot gases flowing in the heat transfer channels.

As yet another alternative, if the catalyst used for the first reaction (steam methane reforming) is also suitable for use for the second reaction (combustion) and vice versa, then the ducts could be disconnected from the reactor block, say the reactor block 12a, and then reconnected to the other set of channels. In this example this would entail supplying the steam/methane mixture to the second set of channels 16, and supplying the air/methane mixture to the first set of channels 15. If the catalysts are not suitable for both reactions, then the catalysts may be removed from the channels 15 and from the channels 16, and inserted into the other set of channels. This would typically involve removing partially or fully spent catalysts and inserting fresh catalysts. In this example, the channels 15 and the channels 16 preferably have the same dimensions so that the same catalysts will fit into the channels and also so that the reaction volumes provided will be the same after the change as they were before the change. It will again be appreciated that such a change may be made to the other reactor block 12b, either instead of or as well as making the change to the reactor block 12a.

In this example, as another alternative, both the reactor blocks 12a and 12b may be disconnected, and exchanged in position, and then reconnected so that the first stage of the reaction takes place in the reactor block 12b and the second stage in the reactor block 12a. This may involve moving the reactor blocks themselves, or leaving the reactor blocks in place and changing the flow ducts.

It will be appreciated that the plant may include the ducts suitable for bringing about the reversal of flow direction, so that it is only necessary to change the position of valves. This is illustrated in FIG. 1 in relation to the flow of methane and steam into the first stage reactor block 12a. A shut-off valve 30 may for this purpose be provided in the inlet duct leading to the first flow channels 15, and a shut-off valve 32 be provided in the duct 20 leading from the outlet of the first flow channels 15. An inlet bypass duct 34 (shown as a broken line) communicates between upstream of the shut-off valve 30 and upstream of the shut-off valve 32, and an outlet bypass duct 36 (shown as a broken line) communicates between downstream of the shut-off valve 30 and downstream of the shut-off valve 32. Both the inlet bypass duct 34 and the outlet bypass duct 36 are also provided with shut-off valves 35 at both ends. In the initial mode of operation both the shut-off valves 30 and 32 are open, whereas the shut-off valves 35 are all closed. The methane and steam mixture therefore flows through the reactor block 12a along the flow channels 15 from left to right as described previously. When the flow direction is to be reversed, the shut-off valves 30 and 32 are both closed whereas the shut-off valves 35 are all opened. In this case the methane and steam mixture flows along the inlet bypass duct 34, and then along the flow channels 15 from right to left, and then along the outlet bypass duct 36. It will be appreciated that a similar arrangement of inlet and outlet bypass ducts and shut-off valves may also be provided for the combustion gases provided to the first stage reactor block 12a. It will also be understood that the second stage reactor block 12b may be modified with such bypass ducts and shut-off valves in the same way.

After making such a change in a reactor block, say block 12a, then when the module 10 is brought back into use the thermal stresses will affect different portions of the reactor block 12a. Hence the portion of the reactor block that is subjected to the greatest thermal stress is changed from an initial portion to a different portion, so if any degradation of the reactor block results from the stress, further degradation of the initial portion is suppressed and subsequent degradation occurs at the different portion. Hence the operational life of the reactor block can be increased.

Although the invention has been described above in relation to a two-stage steam methane reforming module it will be appreciated that it is applicable to any chemical reactor in which there are reaction channels and heat transfer channels, whether single or multi-stage. By way of example the invention would be applicable to a partial oxidation reactor, or an autothermal reforming reactor, which are alternative reactors for producing synthesis gas. It would also be applicable to a reactor for performing Fischer-Tropsch synthesis. This is an exothermic reaction carried out at elevated pressure, and in this case the first channels contain a catalyst but the second flow channels carry only a coolant. In this case after a period of operation, and with the reactor shut down, a reactor block may be disconnected from its associated inlet and outlet ducts, and the reactor block turned around; or the inlet and outlet ducts altered while leaving the reactor block in its original position; in either case the changes ensure that the flow direction of the reactants through the reactor block is reversed. As an alternative, if Fischer-Tropsch synthesis is performed using a reactor module containing two reactors in series, so that the synthesis occurs in two stages, then after a period of operation, and with the reactor module shut down, the reactors forming the module may be exchanged.

In a further alternative, particularly applicable to a plant that contains a plurality of reaction modules that operate in parallel, the flow of reactants supplied to one module may be altered, for example being increased by 20%; at the same time the flow of reactants to another parallel module might be decreased by 20%, so that the overall flow through the plant is not changed. Such changes will affect the temperature distribution within the reactor or reactors of each module. At a subsequent time, for example after a period of a week or a month, the flow of reactants to the one module might be decreased and the flow of reactants to the other parallel module might be increased, so that again the temperature distribution within the reactor of reactors of each module is altered. By repeatedly making such changes the deleterious effects of the thermal stresses are mitigated.

1. A method of operation of one or more chemical reactors, wherein each chemical reactor defines first flow channels for steam/methane reforming reaction in proximity to second flow channels for combustion, so there is heat transfer from the combustion reaction in the second flow channels to the steam/methane reforming reaction in the first flow channels, and each chemical reactor is provided with fluid connections for bringing about flows of respective fluids through the first flow channels and the second flow channels, wherein the method comprises modifying the flows of fluid through the first flow channels or the second flow channels or both, so as to change the temperature distribution within the or each reactor, while the chemical reaction process that takes place in the chemical reactors remains the same.

2. A method as claimed in claim 1 wherein the method comprises the steps of shutting down the flows of fluids through at least one of the first flow channels and the second flow channels, and then changing the fluid connections, and then reopening the fluid connections.

3. A method of operation as claimed in claim 2 wherein the chemical reactor forms part of a plant that includes a plurality of other chemical reactors, and the steps of the method are performed during maintenance or shutdown of the plant, or during maintenance or shutdown of the chemical reactor while the plant continues to operate.

4. A method of operation as claimed in claim 2 wherein the step of changing the fluid connections to the reactor comprises the steps of disconnecting the reactor from inlet and outlet flow connections, and then turning the reactor around, and then reconnecting the inlet and outlet flow connections so that the flow direction through the reactor is reversed for either the first flow channels or the second flow channels or both.

5. A method of operation as claimed in claim 2 wherein the flow connections comprise ducts and wherein the step of changing the fluid connections to the reactor comprises the steps of disconnecting the reactor from inlet and outlet ducts, then altering the ducts, and then reconnecting the ducts so that the flow direction through the reactor is reversed in either the first flow channels or the second flow channels, or both.

6. A method of operation as claimed in claim 2 wherein the flow connections comprise ducts, and the reactor is provided with bypass ducts and shut-off valves, wherein the step of changing the fluid connections to the reactor involves opening or closing shut-off valves communicating with the bypass ducts so as to reverse the flow direction through the reactor.

7. A method of operation as claimed in claim 2 wherein the chemical reaction process is performed using two reactors arranged in series, and wherein the step of changing the fluid connections involves exchanging the positions of the two reactors.

8. (canceled)

9. (canceled)

10. (canceled)

11. A method as claimed in claim 1 wherein the chemical reactors form part of a plant that includes other chemical reactors, and wherein the chemical reactor to which the flow modification is applied is connected either in parallel or in series with other chemical reactors of the plant.

12. A chemical plant including one or more chemical reactors, along with means for performing a method as claimed in claim 1.

13. (canceled)

14. A module comprising a first reactor and a second reactor, each reactor comprising a stack of plates that are bonded together, and defining first flow channels for a chemical reaction process in proximity to second flow channels for heat transfer, the first flow channels and the second flow channels being arranged alternately in the stack, and the module also comprising ducts configured to take outputs from the first reactor to provide inputs to the second reactor, and bypass ducts and valves configured to take outputs from the second reactor to provide inputs to the first reactor, such as to modify the flows of fluid through the first flow channels or the second flow channels or both, so as to change the temperature distribution within each reactor, while the chemical reaction process that takes place in each of the chemical reactors remains the same.

15. A method as claimed in claim 1 wherein each chemical reactor comprises a stack of plates that are bonded together, the first flow channels and the second flow channels being arranged alternately in the stack.
